(19) United States
(12) Patent Application Publication
(10) Pub. No.: US 2011/0081405 A1
(43) Pub. Date: Apr. 7, 2011

(54) Zeolite Formulation And Use Thereof For The Prevention And Therapy Of Diseases Caused By Infections With Herpes Simplex Virus Type 1 And Type 2

(76) Inventors: Zorka Mikloska, Oyster Bay (AU); Boris Subotic, Zagreb (HR); Mladen Zinic, Zapresic (HR)

(21) Appl. No.: 12/896,447

(22) Filed: Oct. 1, 2010

Related U.S. Application Data
(63) Continuation of application No. PCT/HR2009/000012, filed on Apr. 3, 2009.

(30) Foreign Application Priority Data
Apr. 3, 2008 (HR) P20080158A

(51) Int. Cl. A61K 9/127; C01B 39/02; A61K 33/06; A61K 9/14; A61P 31/12

(52) U.S. Cl. 424/450; 423/700; 424/684; 424/489

(57) ABSTRACT

Antiviral medicine (compound) presented here is comprised of an active substance, a carrier of active substance and additives, and it is used for the prophylaxis, therapy and/or pre-respectively post-treatment of diseases caused by the infection with Herpes simplex virus type 1 and/or 2.

The active substance is represented by the synthetic zeolites in pure form, with defined crystal structures and chemical compositions. The active substance (zeolite) has a crystal size of 0.1-10 μm and specific surface of 400-1200 m²/g. The sodium ions can be partially or completely exchanged with other cations, e.g. K⁺, Ag⁺, NH₄⁺, Ca²⁺, Mg²⁺, Mn²⁺, Zn²⁺, Cu²⁺, Fe²⁺ and Fe³⁺ in active substance of this antiviral compound.

The carrier of active compound is the organic gel, water, oil, cream, liposome and liposome-based systems with regular and/or prolonged activity. The weight ratio between the active substance and the carrier is 5×10⁻⁹-10⁻³, i.e. 5 ng to 1 mg of active substance per one gram of the carrier. The additives used are vitamines: Vitamin C (0-20 wt. %), vitamin E (0-0.01 wt. %), vitamin A (0-1 wt. %) and vitamin D3 (0-1 wt. %).

The efficacy of this preparation was demonstrated in in vitro experiments of the inhibition of HSV-1 and HSV-2 plaques formation in keratinocyte monolayers that were preincubated, coincubated or postincubated with the virus at the different time points (by up to 92%).

### TECHNICAL FIELD

The present invention relates to the formulation and the use of preparation for prevention and therapy of diseases caused by infections with Herpes simplex virus type 1 and type 2.

### BACKGROUND AND PRIOR ART

### The Infection with Herpes Simplex Viruses

There are two types of Herpes simplex viruses: HSV-1 usually infects orolabial and nasal region, while HSV-2 lesions predominate in genital areas. Despite the extensive research in last fifty years, there is not yet an efficient treatment developed against genital herpes. Genital herpes is together with infections caused by Human papilloma virus (HPV), chlamydia and Human immunodeficiency virus, widely distributed sexually-transmitted disease. The virus is a main non-traumatic cause of corneal blindness in developed countries; it also may cause neonatal encephalitis with a high mortality rate. The presence of HSV genital lesions is also a risk factor for acquisition or transmission of HIV through unprotected sexual contact (L. Gwanzura, W. McFarland, D. Alexander, R. L. Burke, D. Katzenstein. J Infect Dis. 1998, 177, 481.).

Among various populations, between 20-60% sexually active men and women in the world have antibodies for HSV-2, and between 40-90% antibodies for HSV-1 (L. Corey, H. G. Adams, Z. A. Brown, K. K. Holmes. Ann Intern Med. 1983, 98, 958.).

Almost 80% of infected people have a few, or have not at all the symptoms of the viral shedding, although it is probable that most of the infected people do have genital herpetic lesions, but they fail to recognize them (A. Wald, J. Zeh, S. Selke, T. Warren, A. J. Ryncarz, R. Ashley, J. N. Krieger, L. Corey. N Engl J Med 2000, 342, 844.).

In general, the prevalence is higher in developing countries than in developed, in urban territories than in rural ones and among the women rather than men, especially between younger population (http://www.who.int/docstore/hiv/herpes_meeting/004.htm).

In the USA, prevalence of infected people is higher than in Europe, almost every fourth person in the USA, i.e. 22% of the adult population, has genital herpes. Infection with HSV-2 is common in sub-saharian Africa, where HIV infection is highly prevalent.

After initially infecting skin or mucosa in the episode of primary infection, virus is transported to the neurons of dorsal root ganglia, where, after short replication, establishes life-long latency. Upon appropriate stimuli, the HSV reactivates, and is transported back in anterograde fashion to reinfect originally infected dermatome, causing an episode of recurrent herpes. Such recurrences can be spontaneous or associated with different external stimuli such as physical or emotional stress, fever, exposure to UV light, tissue and/or nerve damage, or immune suppression. The danger for horizontal spread is in the predominant asymptomatic lesions of skin/mucosa. In an immuno-competent host, recurrent HSV is a self-limiting disease where CTL, cytokines of Th1 type, and, to lesser extent neutralizing antibodies, resolve lesion with time (A. L. Cunningham, Z. Mikloska. Herpes. 2001, Suppl 1:6A.).

In epidermal cells infected with HSV, virus induces some morphological changes, unlike after HPV infection. Any morphological detectable changes in the host cell due to infection with viruses are known as a virus caused cytopathic effect (CPE). CPE may serve as a prognostic tool for monitoring virus infection.

### Treatments for the Disease Caused by HSV Infection

Life-long latency, the immune evasion by the virus and the ability of virus for quick spread out of reach of immunity have all being obstacles to efficiently combat virus infection. Main goal in therapy against genital herpes infections is in the reduction of duration and severity of primary and recurrent infections, thus converting the symptomatic into asymptomatic lesions with time.

There is no efficient vaccine against genital herpes developed yet; the only viable treatment is by antiviral drugs. Unfortunately, antiviral therapy against HSV is only a short-term option since it is only partially efficient and expensive in most countries, when compared with cheap generic drugs. Also, frequent use of high doses of antiviral agents can have an adverse effect in patients, also, with time, antiviral agents become inefficient to the resistant viral strains (H. B. Gershengorn, G. Darby, S. M. Blower S M. BMC Infect Dis. 2003, 3, 1.).

Antiviral agents inactivate virus or inhibit development and the ability of virus to replicate, thus giving time to immune system to react (R. Whitley. Herpes. 2006, 13, 53.).

There are two main types of antiviral agents used: antiviral agents used against recurrent herpes labialis caused mainly by HSV-1 (S. L. Spruance, J. D. Kriesel. 2002, Herpes, 9, 64.), and antiviral agents used against genital herpes caused by HSV-2 (R. Gupta, A. Wald. Expert Opin Pharmacother. 2006, 7, 665). Three antiviral agents, most commonly used today, either orally or intravenously, are: acyclovir (ACV) (G. B. Elion. Am J Med, 1982. 73, 7.), valacyclovir (VAL), and famcyclovir (FAM) (R. Hamuy, B. Berman. Drugs Today. 1998, 34, 1013.). They act similarly, by inhibiting the replication of the virus. Upon entering the cell, the antiviral becomes phosphorylated and acts as a nucleoside analogue, thus terminating replication of viral DNA. Viral thymidine kinase is used for the first phosphorylation step (C. S. Crumpacker. Am J Med. 1992, 92, 3S.). The disadvantages of those antivirals are: a) a relatively low biavalibility of the drug (20% for acyclovir, 55% for valacyclovir, 77% for famcyclovir, b) a need to be administered couple of times per day (5 for acyclovir and 2-3 for valacyclovir), and c) a generation of thymidine kinase-lacking viral mutants, which are resistant to the drug (M. H. Schmid-Wendtner, H. C. Korting. Skin Pharmacol Physiol. 2004. 17, 214.).

The patent application titled <<TMAZ as an antiviral agent and use thereof>> (Publication number: WO2007054085, Date of publishing 2007-05-18; inventors Lelas Tihomir, proposers Ljubicic Mijo, Ivkovic Slavko, Lelas Tihomir, International classification: A61K33/06; A61K35/20; A61K35/64; A61P31/12; A61K33/06; A61K35/20; A61K35/56; A61P31/00; European classification: A61K33/06; A61K35/20; A61K35/64; A61K45/06, Number of application: WO2006DE02008 20061110, Priority number(s): DE200510054306 20051111), describes the use of TMAZ (tribomechanically activated clinoptilolite), i.e. a mixture of natural mineral material, mostly clinoptilolite, with the particle size <100 nm, preferably <50 nm, with the addition of mostly propolis and/or colostrum, as a antiviral agent. In this patent application, the use of <<tribomechanically treated>> natural clinoptilolite was described. It is well known that the portion of the zeolite (clinoptilolite) 30-90 w %, and therefore the chemical composition in natural material, depends on a deposit; also, the properties of zeolite mentioned here could vary depending on the exploiting site (the position, depth, etc.) at the same deposit. Therefore, it is unattainable to maintain constant mineral and chemical composition of naturally occurred clinoptilolite. A particular problem is a variable and unpredictable cationic content, including the existence of heavy ions. Furthermore, because minerals appear in the form of clumps, natural clinoptilolite has to be fragmented to get the desired size. Despite the claims of the authors of this patent application, the main result of any mechanical treatment is the separation of zeolite (clinoptilolite) from impurities (clay, different crystal and amorphous types of silicium dioxides, the other types of zeolites), and eventually, the de-segregation of crystal aggregates of clinoptilolite and other mineral materials. Because of this, the particulate properties of mechanically treated natural clinoptilolite are undefined and unpredictable. Therefore, the argument that the particle size of mechanically treated clinoptilolite is in the nanometer range (>100 nm, preferably >50 nm), is somewhat dubious, moreover, the authors do not offer evidence to support this argument. In fact, it is well known that the crystal size of natural clinoptilolite lies in the micrometer-size range. Moreover, the use of intense mechanical forces causes destruction of aluminosilicate structure of zeolite framework, and thus its transformation in amorphous state, followed by the loss of specific structural characteristics of zeolite (C. Kosanović, J. Bronić, B. Subotić, I. \check{S}mit, M. Stubi\check{c}ar, A. Tonejc, T. Yamamoto, Zeolites 1993, 13, 261.). In addition, the mechanical treatment causes aggregation of newly formed amorphous particles and their enlargement, rather than diminution. Finally, the effect of use of those preparations in very high concentrations on patients with diagnosed primary and secondary immune disorders (daily doses of 6 to 16 tablets containing at least 1.8 g to 4.8 g of TMAZ) was described in this patent application. Three main disadvantages of the treatments described here, are: a) an unknown ratio of chemical compounds in a mixture containing natural zeolite clinoptilolite, so that the variation in the chemical composition cannot be avoided; b) a questionable purity of those preparations, and c) a relatively high doses of preparation used. The antiviral activity of TMAZ in combination with propolis and colostrum on HIV infected patients was described as an increase in the number of neutralizing antibodies and a decrease of viral titre in patient's blood. However, the <<adsorption of the virus was observed>> when the anti HSV activity by TMAZ was studied. If method for testing of anti HSV activity by TMAZ was indeed by measuring the adsorption of virus to the cells, the opposite is correct: higher antiviral activity corresponds to a lower viral adsorption to the cells treated with antiviral.

TABLE 1
Comparing of natural and synthetic zeolites

| Characteristics | Natural clynoptilolite | Synthetic zeolites |
|---|---|---|
| Structure | Not constant, dependent on the | Constant and defined for specific |
| | deposit, and also other minerals and | type of zeolite |
| | nonminerals on the site. | |
| Chemical composition | Not constant, dependent on the | Constant and defined for specific |
| | deposit, and other minerals and | type of zeolite |
| | nonminerals on the site. | |
| Particulate properties | Dependent on the choice of natural | Dependent on the condition of |
| | material. The size of particles | synthesis, and therefore it could be |
| | mentioned in patent application | adjusted according to the conditions |
| | above (<100 nm or <50 nm) is not | of synthesis |
| | supported by evidence. | |
| Daily doses | 10.8-76.8 g | 5 × 10⁻⁹-1 × 10⁻³ g |
| Mode of therapy | Oral | Topical through the mouth, genital |
| | | or anal mucosa |
| Indications | Many diseases and pathological | Symptoms of in vitro infections with |
| | disorders caused by infectious and | Herpes simplex viruses type 1 |
| | noninfectious agents | and/or 2 |
| Effects | Acting as immunomodulator. | Inhibition of viral spread in vitro |
| | Preventing viraemia in patients.. | |

### DEFINITION OF THE INVENTION

The present invention which comprises
(i) active components(s) which are microcrystalline particles of zeolite(s) having defined crystal structure, chemical composition and particle size
(ii) an inert, non-toxic carrier of the active component(s), and
(iii) additives and vitamins,
   is related to the preparation and the use of antiviral preparation in the forms of powder, water or oil suspension, tablets, plaster, patch, cream, suppository, gel, inhalates, aerosol, spray, droplets, liposomes and carriers similar to liposome, with normal or extended (prolonged) activity, for the prevention and therapy of diseases caused by infections with HSV type 1 and 2.

### BRIEF DESCRIPTION OF FIGURES

The objective of the invention is represented by the enclosed figures, which show:

FIG. 1. Schematic presentation of the linking of SiO4 and AlO4 tetrahedra in zeolite framework structure.

FIGS. 2(a), 2(b), 2(c), 2(d), 2(e), 2(f). Structures of the unit cells of (a) zeolite A (LTA type), (b) faujsaite (zeolites of X and Y; FAU types), (c) zeolite P (GIS type), (d) mordenite (MOR type), (e) clinoptilolite (HEU type) and (f) zeolites YSM-5 and silicalite-1 (MFI type).

FIGS. 3(a), 3(b), 3(c), 3(d), 3(e), 3(f). Photomicrographs of (a) zeolite A (LTA type), (b) hydroxysodalite, (c) faujsaite (zeolites of X and Y; FAU types), (d) zeolite P (GIS type), (e) mordenite (MOR type), and (f) zeolites YSM-5 and silicalite-1 (MFI type) illustrating the morphological characteristics of the selected types of zeolites.

FIGS. 4(a) and (b). Photomicrographs of zeolite A crystals obtained under different synthesis conditions illustrating their morphological characteristics.

FIG. 5(a) and (b). Photomicrographs of zeolite ZSM-5 crystals obtained under different synthesis conditions illustrating their morphological characteristics.

FIG. 6. Graphs showing thermogravimetric (A) and differential thermogravimetric (B) curves of one of the zeolites synthesized by the procedures described in the Working example 1, and modified by ion-exchange procedure described in the Working procedure 2.

FIGS. 7(a), 7(b), 7(c), 7(d), 7(e). Graphs showing crystal size distributions of (a) zeolite X, (b) hydroxysodalite, (c) zeolite A, (d) zeolite P and (e) mordenite synthesized by the procedures described in the Working procedure 1 and measured by the method described in the Working procedure 8.

### DETAILED DESCRIPTION OF THE INVENTION

There is no effective vaccine against HSV-1 and HSV-2 developed yet. All available antiviral agents operate using the same mechanism (the inhibition of virus replication) and they are effective only when applied in high concentrations (R. Hamuy, B. Berman B. Drugs Today. 1998, 34, 1013.).

Those antiviral agents can cause side-effects during repeated application, and thereafter are not efficient against the newly-formed resistant strains of the virus (C. Scieux, A. Bianchi. Pathol Biol (France). 1993, 41, 172.). In addition, the existing antiviral agents are not economic, especially in the comparison with generic drugs.

Our intention was to develop a new antiviral drug, efficient in low concentrations and which efficiency does not decrease with time. The solution of the problem has been found in the application of zeolite(s), which retard(s) entering of viruses into the cell, and at the same time, inhibit(s) their propagation.

### Structure, Chemical Composition and Properties of Zeolites

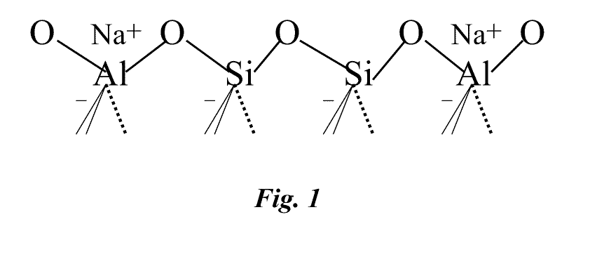

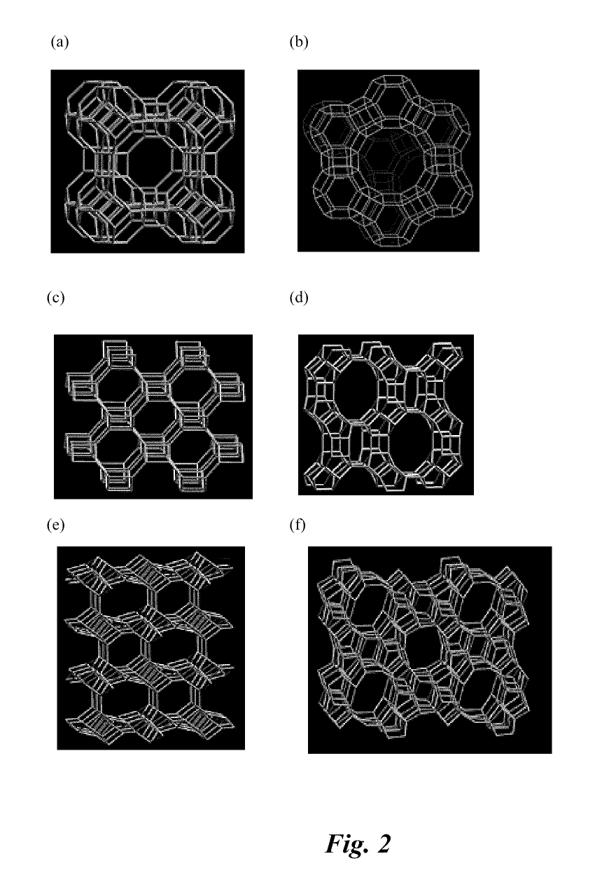

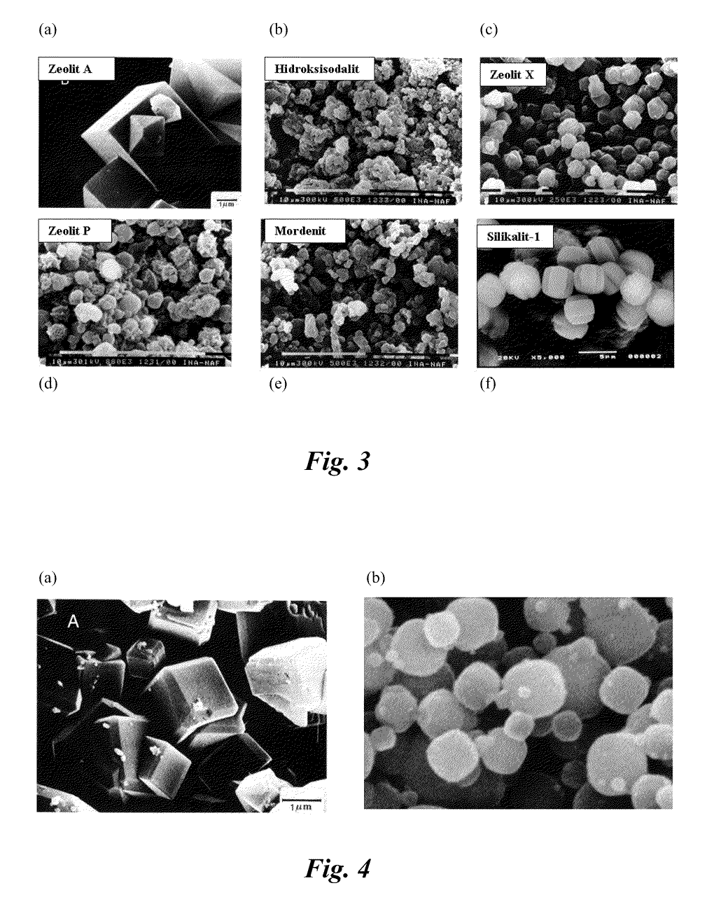

Zeolites or molecular sieves are hydrated natural and synthetic aluminosilicate compounds with exceptional framework structure formed of SiO₄ i AlO₄ tetrahedrons linked by common oxygen atoms (D. W. Breck, J. Chem. Educ 1964, 41, 678.), as it is schematically presented in FIG. 1. Negative charge of the aluminosilicate structure caused by isomorphous substitution of four-valent silicon with three-valent aluminium is compensated by hydrated cation (Na⁺, K⁺, Ca²⁺, Mg²⁺ i etc.) (D. W. Breck, J. Chem. Educ 1964, 41, 678.) However, in real zeolite framework, SiO₄ i AlO₄ tetrahedrons do not form one-dimensional chain-like structures as it is in a simplified way presented in FIG. 1, but two- and three-dimensional structure building units (SBU-s); their combination results in the formation of three-dimensional framework structures characteristic for zeolites (R. Szostak, Molecular Sieves: Principles of synthesis and Identification, Van Nostrand Reinhold, New York, 1989; J. B. Nagy, P. Bodart, I. Hannus, I. Kiricsi, Synthesis, Characterization and Use of Zeolitic Microporous Materials, DecaGen Ltd., Szeged, Hungary, 1998). Specificity of zeolite structure, unique in the relation to other aluminosilicate materials as well as to other crystalline materials, manifests in the existence of the structural voids mutually connected with “windows” and/or channels of defined size and shape. However, as opposed to other porous material characterized by a random distribution of pores, size and shape of the voids, “windows” and channels of zeolites as well as their mutual relationships are constant and exactly defined as the structural parameters of the given type of zeolite (W. H. Meier, D. H. Olson, Atlas of Zeolite Structure types, Publ. by the Structure Commission of the International Zeolite Association, (1978).), as can be seen in FIG. 2 which shows the examples of the unit cells of the most common used types of zeolites. The zeolites prepared by the “standard” synthesis procedures (H. Robson, Verified Syntheses of Zeolitic Materials, 2nd Edition, International Zeolite Association, 2001.) usually appear in the form of fine white powder having the particle (crystal) size in the micrometer-size range (B. Subotić, J. Bronić, in: S. M. Auerbach, K. A. Carrado and P. K. Dutta (Eds.), Handbook of Zeolite Science and Technology, Chp. 5, Theoretical and Practical Aspects of Zeolite Crystal Growth, Marcel Dekker Inc., New York—Basel, 2003, p.p. 129-203.). Crystal size distribution of a given type of zeolite depend on the synthesis conditions (I. Krznarić, T. Antonio, B. Subotić, V. Babić-Ivan\check{c}ić, Thermochimica Acta 1998, 317, 73.), while the crystal shape mainly depends on the structural type of zeolite (see FIG. 3) and in a less extent on the synthesis conditions (see FIGS. 4 i 5) (B. Subotić, J. Bronić, in: S. M. Auerbach, K. A. Carrado and P. K. Dutta (Eds.), Handbook of Zeolite Science and Technology, Chp. 5, Theoretical and Practical Aspects of Zeolite Crystal Growth, Marcel Dekker Inc., New York—Basel, 2003, p.p. 129-203.). Chemical composition of zeolites is usually expressed by a general oxide formula, i.e,

(M_{2/n})O.Al₂O₃.y SiO₂.z H₂O

where n is the charge of cation M, and y ≧2 and z depend on the type of zeolite. “Zeolitic” water arises from the hydration shells of the compensating cations M (D. W. Breck, J. Chem. Educ 1964, 41, 678.; J. B. Nagy, P. Bodart, I. Hannus and I. Kiricsi, Synthesis, Characterization and Use of Zeolitic Microporous Materials, DecaGen Ltd., Szeged, Hungary, 1998.). Hence, the value of z depends on the type of compensating cation, number of cations in the unit cell of zeolite and on the degree of hydration of cation M in the zeolite framework. Heating of zeolites to about 600° C., “zeolitic” water can be irreversible removed without the change of the framework structure (C. Kosanović, B. Subotić, A. \check{C}i\check{z}mek, Thermochimica Acta 1996, 276, 91.). During the cooling down to ambient temperature, zeolite absorbs the same amount of water, i.e., the processes of absorption and desorption are strongly reversible (D. W. Breck, J. Chem. Educ. 1964, 41, 678.; G. T. Kerr, J. Phys. Chem. 1966, 70, 1041.; J. Ciric, J. Colloid Interface Sci. 1968, 28, 315.).

In contact with electrolytic solutions, the cations from zeolite can be reversible exchanged with the zeolite host cations (R. M. Barrer, J. Klinowski, Phil. Trans. 1977, 285, 637.; B. Bi\check{s}kup, B. Subotić, Sep. Sci. Technol. 1998, 33, 449.; B. Bi\check{s}kup, B. Subotić, Phys. Chem. Chem. Phys. 2000, 2, 4782.; B. Bi\check{s}kup, B. Subotić, Sep. Sci. Technol. 2000, 35, 2311.; B. Bi\check{s}kup, B. Subotić, Sep. Purif. Tehnol. 2004, 37, 17.; B. Bi\check{s}kup, B. Subotić, Sep. Sci. Technol. (2004), 39, 925.). In the equilibrium condition,

zB×A^{zA} (aq)+zA×B^{zB} (s) [symbol] zB×A^{zA} (s)+zA×B^{zB} (aq)

where zA i zB are charges (“valencis”) of the exchangeable cations A i B, and aq i s denote the solution and solid phase (zeolite), respectively.

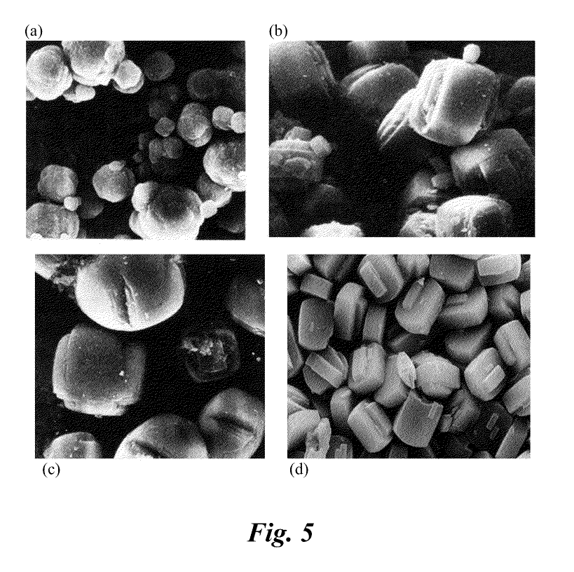

The mentioned chemical and structural properties of zeolites are the basis for their wide application as selective cation exchangers, absorbents, molecular sieves, catalysts, etc. [E. M. Flanigen, in: Proc. Fifth. Int. Conf. Zeolites (Ed. L. V. C. Rees), Heyden, London-Philadelphia-Rheine, 1980, p. 760.; B. Subotić, J. Bronić, A. \check{C}i\check{z}mek, T. Antonić, C. Kosanović, Kem. Ind. 1994, 43, 475) Annually, millions metric tones of zeolites are used in the manufacturing of washing formulations, hundreds thousand metric tones of zeolites are used in the oil processing and petrochemical industry, and also application in other areas, including actual and potential application of zeolites in agriculture, cattle-breeding, fish-farming, medicine and pharmacology is in progression. (A. J. Ramos, E. Hernandez, Animal Feed Sci. Technol., 1997, 65, 197.; H. Eriksson, Biotechnology Techniques, 1998, 12, 329.; F. A. Mumpton, J. Nat. Acad. Sci., 1999, 96, 3463.; K. Pavelić, B. Subotić, M. \check{C}olić, Stud. Surf Sci. Catal. 2001, 135, 170.; K. Pavelić et al. Journal of Molecular Medicine-Jmm. 2001, 78, 708.; K. Pavelić, M. Katie, V. \check{S}verko, T. Marotti, B. Bo\check{s}njak, T. Balog, R. Stojković, M. Rada\check{c}ić, M. Colić, M. Poljak-Bla\check{z}i, J. Canc. Res. Clin. Oncol. 2002, 128, 37.; N. \check{Z}arković, K. \check{Z}arković, M. Kralj, S. Borović, S. Sabolović, M. Bla\check{z}i-Poljak, A. Cipak, K. Pavelić, Anticancer Research. 2003, 23, 1589.; M. Grce, K. Pavelić, Microporous Mesoporous Mater. 2005, 79, 165.; M. Katie, B. Bo\check{s}njak, K. Gall-Tro\check{s}elj, I. -Dikić, K. Pavelic, Frontiers in Bioscience (2006), 11, 1722.). The mentioned potential applications of zeolites in medicine and pharmacology are more or less related to natural zeolites, mainly natural clinoptilolite. Since the application of natural zeolites is encountered with numerous drawbacks such as (a) variability of chemical and mineralogical compositions (purity) and their dependence on the deposit, (b) variable and unpredictable cationic composition and (c) bad control of particulate properties, there is a strong tendency for a substitution of natural zeolites with synthetic ones, for their application in medicine and pharmacology; synthetic zeolites can be obtained in pure, fully crystalline form having defined crystal structure and chemical composition. In addition, essential properties of zeolites such as type (structure), chemical composition (including cationic one), crystal size (from micrometer to nanometer size range), and in many cases, the crystal shape among the same structural type (see FIGS. 4 and 5) can be changed in an controlled way by the controlling of the synthesis conditions. Due to the mentioned reasons, the present invention is based on the application of both natural and synthetic zeolites.

### Example 1

### Synthesis of Zeolites

The zeolites, used as the active antiviral agents in the present invention, are synthesized by a series of procedures as follows: (A) Preparation of aluminosilicate precursor (hydrogel) by mixing together the alkaline aluminate and silicate solutions and/or by mixing together the amorphous silica and alkaline aluminate solutions, with or without addition of needed additives (inorganic salts, organic templates, pore fillers, modifiers etc.) at determined temperature Tᵣ≦Tₚ≦T_{c}, where Tₚ is the temperature of hydrogel preparation, Tᵣ is the ambient (room) temperature, and T_{c} is the temperature of crystallization. (B) “Ageing” of hydrogel at the temperature Tᵣ≦Tₐ<T_{c}, where Tₐ is the temperature at which the hydrogel is “aged” during the time tₛ, before crystallization. (C) Transformation of the solid phase of hydrogel (amorphous aluminosilicate) into the crystalline phase (zeolite), i.e. process of crystallization at the elevate temperature T_{c}, until the amorphous aluminosilicate precursor is completely transformed to the crystalline phase (zeolite). (D) Separation of the solid phase (zeolite) from the liquid phase (supernatant) by vacuum filtration and/or centrifugation, after the process of crystallization is completed. (E) Washing (rinsing) of zeolite with demineralized water to remove the components contained in supernatant, from the surface of zeolite crystals. (F) Drying of the washed (rinsed) product (zeolite)—usually, at 105-110° C. for 1-24 h. (G) Thermal treatment in order to decompose organic template(s) and/of pore fillers/modifiers (only for the synthesis procedures which include the mentioned organic additives).

The synthesis conditions in accordance with the procedures (A)-(F) for different types of zeolites are determined in accordance with the verified “standard” procedures of their syntheses [H. Robson, Verified Syntheses of Zeolitic Materials, 2nd Edition, International Zeolite Association, 2001.]. All the synthesized zeolites are obtained in the form of fine white powder having the chemical composition (M_{2/n})O.Al₂O₃.y SiO₂.z H₂O and crystal size in micrometer range (0.5-10 micrometers) (see Working example). Here, Me=Na (most often), K, Ca i Mg and their mixtures in different proportions, respectively, y=2-100 and z=0.01-4.65.

### Example 2

### Modification of Zeolites by Ion Exchange

Intention of the modification by ion exchange is obtaining of zeolites in defined, mono-ionic form. For this purpose, determined amounts (10-50 g) of synthetic zeolites (prepared as described in the Working example 1) and/or their natural analogues (only for the synthetic zeolites that have natural analogues) is suspended (dispersed) in 1000 ml 0.001-5.0 M solution of Meⁿ⁺ ions (Meⁿ⁺=Na⁺, K⁺, Ag⁺, NH₄⁺, Ca²⁺, Mg²⁺, Mn²⁺, Zn²⁺, Cu²⁺, Fe²⁺, Fe³⁺) at 20-80° C. The obtained suspension of zeolite in solution of ions is stirred for 10-300 min at given working (exchange) temperature (20-80° C.). Thereafter, the solid phase (zeolite) is separated from the solution by vacuum filtration and/or centrifugation, and zeolite is washed (rinsed) by demineralized water, until the reaction to the exchangeable ions in filtrate (centrifugate) is negative. The washed (rinsed) modified zeolite is dried at 105-150° C. for 1-24 h.

### Product Characterization

The products (zeolites synthesized by the procedures described in the Working example 1 as well as natural and synthetic zeolites modified by ion exchange as described in the Working example 2) are characterized by powder X-ray diffractometry (XRD), Fourier transform infrared spectroscopy (FTIR), absorption atomic spectroscopy (AAS), scanning-electron microscopy (SEM), thermal analysis (thermogravimetry, TG; differential thermogravimetry, DTG), crystal size distribution analysis (CSD) and surface analysis (determination of the specific surface area), before and after modification.

### Example 3

### XRD

X-ray diffraction patterns of the zeolites synthesized by the procedures described in the Working example 1 and modified by ion exchange as described in the Working example 2 were taken by a Philips diffractometer with CuK_{α} radiation in the Braggs angles range 2θ=10°-46°. All the samples of zeolites of given types, synthesized by the procedures described in the Working example 1 and modified by ion exchange as described in the Working example 2 are fully crystalline, without admixtures of other types of zeolites and/or amorphous phase.

### Example 4

### FTIR

Infrared spectra of the zeolites synthesized by the procedures described in the Working example 1 and modified by ion exchange as described in the Working example 2 were taken by the KBr wafer technique using FTIR spectrometer System 2000 FT-IR (Perkin-Elmer). All the samples of zeolites of given types, synthesized by the procedures described in the Working example 1 and modified by ion exchange as described in the Working example 2, exhibit the IR spectra characteristic for the types of zeolites previously determined by XRD.

### Example 5

### Chemical Analysis

Chemical analysis of the zeolites synthesized by the procedures described in the Working example 1 and modified by ion exchange as described in the Working example 2 is performed as follows: A determined amount of zeolite is dissolved in diluted nitric acid. Such prepared solution was diluted by demineralized water to the concentration level available for measuring the concentration of aluminium, silicon and corresponding cation by AAS. Acid stable zeolites are fused with a mixture of sodium carbonate and sodium tetraborate. The obtained solid is dissolved in diluted hydrochloric acid. Such prepared solution was diluted by demineralized water to the concentration level available for measuring the concentration of aluminium, silicon and corresponding cation by AAS. Atomic absorption spectrometer 3030B (Perkin-Elmer) is used for measuring of concentrations of aluminium, silicon and cation in the mentioned solutions.

### Example 6

### Thermal Analysis (Thermogravimetry, TG and Differential Thermogravimetry, DTG)

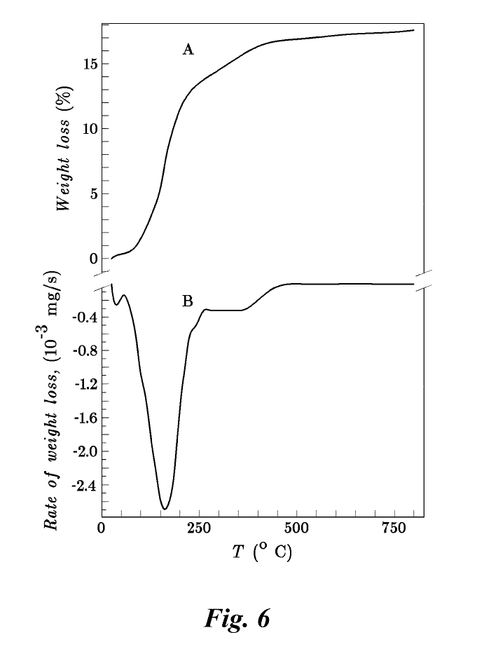

Thermogravimetric (TG) and differential thermogravimetric analyzes of the zeolites synthesized by the procedures described in the Working example 1 and modified by ion exchange as described in the Working example 2 are performed by thermogravimetric apparatus TA 4000 System (Mettler-Toledo). Rate of heating in the nitrogen atmosphere is 10 K/min. Depending in the type of zeolite ant the present cation, content of water is 0.75-27 wt. %. FIG. 6 shows the dependence of the percentage of the weight loss (FIG. A) and the rate of desorption of “zeolitic” water (rate of weight loss, FIG. B) from one of the zeolites synthesized by the procedures described in the Working example 1 and modified by ion exchange as described in the Working example 2.

### Example 7

### SEM

Scanning-electron micrographs of the zeolites synthesized by the procedures described in the Working example 1 (see also FIGS. 3-5) are made by the scanning-electron microscope SEM 515 (Philips).

### Example 8

### Measuring of the Crystal Size Distribution (CSD)

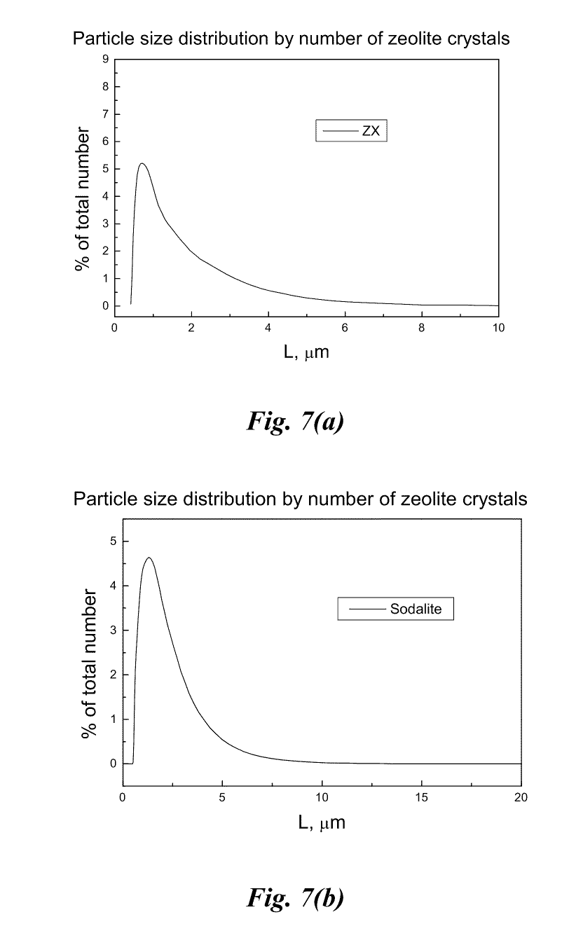

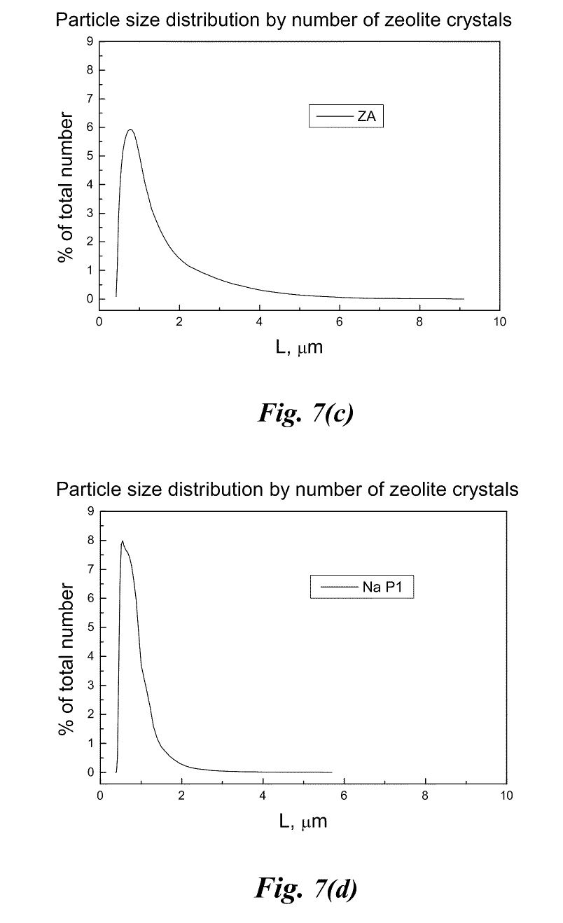

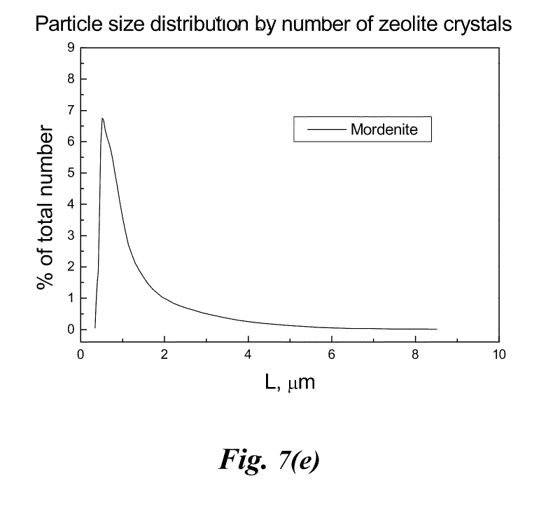

Crystal size distributions (CSD) of the zeolites synthesized by the procedures described in the Working example 1 and modified by ion exchange as described in the Working example 2 are determined by the method of dynamic laser light scattering using the particle size distribution apparatus Mastersize X (Malvern). CSD of some of zeolites synthesized as described in the Working example 1 are shown in FIG. 7.

### Example 9

### Determining of the Specific Surface Area

Specific surface area of the zeolites synthesized by the procedures described in the Working example 1 and modified by ion exchange as described in the Working example 2 are determined by the nitrogen absorption using the apparatus Micromeritics FlowSorb II 2300 instrument. Before measuring, the samples are heated in vacuum at 80° C. for 1 h in order to desorb the surface moisture. Depending on the type of zeolite and average crystal size, the specific surface area of the of the zeolites synthesized by the procedures described in the Working example 1 and modified by ion exchange as described in the Working example is 400-1200 m²/g.

### Example 10

### Testing of Antiviral Activities of Synthetic Zeolites by the Viral Plaque Inhibition Assay—the Antiviral Activity of Zeolites when Coincubated with HSV In Vitro

Primary Cells

Epidermal cell cultures of fibroblasts and keratinocytes were grown in vitro from healthy skin samples, remnants from operations. Fibroblast cultures were grown in Dulbecco modified Eagle's medium (DMEM) containing Glutamax I (L-alanin-L-glutamine) and sodium pyruvate, 4.5 mg/L glucose and pyridoxine, supplemented with 10% fetal calf serum (FCS), 0.25 μg gentamycin and 100 UI/L penicillin. Keratinocytes are grown in a mixture of DMEM, Hams F12 in the 3:1 ratio with the addition of Glutamax I, complemented with 9% of FCS, epidermal growth factor (EGF), insulin, hydrocortisone, triiodothyronine, adenine, and antibiotics. The antiviral effect was determined on primary cells (primary fibroblasts and keratinocytes) grown in vitro from the explants of healthy skin, and also on cell lines: fibroblasts cell line MRC-5, HEp-2 cell line, and Vero cell line.

Cell Lines

MRC-5, Vero, HEp-2, and keratinocyte cell lines were all obtained from the American Type Culture Collection-ATCC, and grown in T-75 flasks at 37° C. in a humidified incubator with 5% CO2.

Viruses

HSV-1 (strain F) and HSV-2 (strain G), both obtained from the ATCC, were propagated in HEp-2 cells and their titres checked in same cells (H. Kirchner, C. Kleinicke, H. Northoff. J. Gen. Virol. 1977, 37, 647.). 0.1, 0.5, 1, 2, 4, or 5 Multiplicity of infection (MOI) of HSV-1 or HSV-2 was used to infect Vero cells, keratinocyte cell lines and MRC-5 cell lines, as described below.

Synthetic Zeolites

Synthetic zeolites: A, Mordenite, P and X were tested in vitro for an anti HSV activity. Mordenite was used in two characteristic size of particles (0.1-0.5 μm and 0.5-5 μm) and other zeolites in 0.5-5 μm particle size. Zeolites were used in the concentrations of 5, 50, 500 ng/ml; 1, 5, 50, 100, 500 μg/ml; 1, 2, 10, 50 mg/ml in growth medium.

The ability of HSV preincubated with zeolite to infect cells permissive to infection (permissive cells) and cell lines was tested in in vitro experiments.

The controls were:

a) cell control: untreated cells (incubated with growth medium only),

b) zeolite control: cells treated with supernatants collected from the cultures preexposed to synthetic zeolite,

c) control with inactivated zeolite: cell treated with supernatants collected from cultures preexposed to inactivated synthetic zeolite (zeolites were previously exposed to dry heat of 900° C. for 2 hours).

d) control with inactivated zeolite and HSV: cells treated with supernatants collected from cultures exposed to inactivated zeolite and HSV,

e) viral control: cells infected with HSV-1 or HSV-2,

f) antiviral control: cells treated with acyclovir (9-[(2-hydroxyetoxy)methyl]guanine), dissolved in growth medium in concentration of 50 μg/ml.

Viral stock containing the HSV-1 or HSV-2 (in MOI described above), diluted in a minimal volume of growth medium containing 5% FCS was used for the coincubation with zeolite for 3-20 hours at 4° C. The pH was maintained at all times to be at around 7.00. The sterile cuvettes containing zeolite+HSV were then centrifuged at 4.000 rpm/30 minutes to remove any particulate content in supernatants. Those supernatants (in 200 μl volume) were then added to the MRC-5, HEp-2, or Vero cell lines (grown in 24 well plate to 85% confluency) for 1 hour at 37° C. Parallel cultures contained cells infected with 200 μl of HSV-1 or HSV-2 under the same conditions as for the addition of supernatants (above). After one hour incubation, the growth medium containing 9% FCS was added, and cells cultivated for another 3 days (to examine for any CPE by light microscopy). Growth medium was then aspirated from the wells, and 100 μl of 0.2% crystal violet dye, diluted in phosphate buffered saline (PBS), was added to each well. After half-hour exposure to the dye, the wells were washed twice with double distilled water, air-dried, and the HSV plaques in cell cultures were counted using Olympus light microscope (magnification of 40×). The CPE produced by the infection of permissive cells was characterised by morphological changes in host cells e.g. cell rounding, swelling, detachment from the surface, formation of syncicia, plaques, caused by the increased number of newly formed viral particles. Plaques formed by HSV destroying cells were easily distinguished from unaffected cell layers. Control with acyclovir, HSV-2 control and cell control were all included in experiments (Table 2 and 3).

TABLE 2
A representative experiment demonstrating the decrease in
percentage of number of HSV-2 plaques in keratinocytes after
the incubation with supernatants containing HSV-2 (0.1 MOI)
and 5 μg/ml of Mordenite, zeolite P, X and A; or 50 μg/ml
of Acyclovir (previously coincubated for 18 hours).

| | 0.1 | 0.5 | 1 | 2 | 4 | 5 |
|---|---|---|---|---|---|---|
| Zeolite/HSV-2 | MOI | MOI | MOI | MOI | MOI | MOI |
| Mordenite | 85 | 83 | 80 | 80 | 71 | 60 |
| P | 62 | 60 | 58 | 53 | 40 | 41 |
| A | 48 | 48 | 42 | 33 | 28 | 26 |
| X | 33 | 36 | 37 | 31 | 25 | 25 |
| Acyclovir (50 μg/ml) | 73 | 72 | 70 | 65 | 48 | 42 |
| Heat inactivated P | 0 | 0 | 0 | 0 | 0 | 0 |
| Heat inactivated | 0 | 0 | 0 | 0 | 0 | 1 |
| Mordenite | | | | | | |
| Heat inactivated A | 0 | 0 | 0 | 0 | 0 | 0 |
| Heat inactivated X | 0 | 0 | 0 | 0 | 0 | 0 |

The particle size of zeolite P, X and A was 0.5-5 μm, and of Mordenite was 0.1-0.5 μm. The antiviral effect of zeolites was calculated as a decrease in the number of HSV-2 plaques developed in cultures treated with supernatants in comparison to viral control. Cell control did not develop any plaques. Number of plaques developed in cells treated with inactivated zeolites and HSV-2 was not statistically different from the viral control.

The highest values of CPE inhibitions were when cells were infected with 0.1 or 0.5 MOI of HSV-2 and incubated with supernatants containing 5 μg/ml of Mordenite, particle size 0.1-0.5 μm. Similar experiments were performed using HSV-1.

TABLE 3
A representative experiment demonstrating the decrease
in number of HSV-1 plaques in keratinocytes after the incubation
with supernatants containing HSV-1 (0.1 MOI) and 5 μg/ml
of Mordenite, zeolite P, X and A; or 50 μg/ml of Acyclovir
(previously coincubated for 18 hours).

| | 0.1 | 0.5 | 1 | 2 | 4 | 5 |
|---|---|---|---|---|---|---|
| Zeolite/HSV-1 | MOI | MOI | MOI | MOI | MOI | MOI |
| Mordenite | 81 | 82 | 79 | 77 | 63 | 55 |
| P | 57 | 59 | 55 | 43 | 42 | 38 |
| A | 42 | 39 | 37 | 28 | 22 | 20 |
| X | 27 | 22 | 25 | 22 | 18 | 20 |
| Acyclovir (50 μg/ml) | 71 | 71 | 61 | 58 | 43 | 36 |
| Heat inactivated P | 0 | 0 | 0 | 0 | 0 | 0 |
| Heat inactivated | 0 | 0 | 0 | 0 | 0 | 1 |
| Mordenite | | | | | | |
| Heat inactivated A | 0 | 0 | 0 | 0 | 0 | 0 |
| Heat inactivated X | 0 | 0 | 0 | 0 | 0 | 0 |

The particle size of zeolite P, X and A was 0.5-5 μm, and of Mordenite was 0.1-0.5 μm. The antiviral effect of zeolites was calculated as a decrease in the number of HSV-1 plaques developed in cultures treated with supernatants in comparison to viral control. Cell control did not develop any plaques. Number of plaques developed in cells treated with inactivated zeolites and HSV-1 was not statistically different from the viral control.

The highest values of CPE inhibitions were when cells were infected with 0.1 or 0.5 MOI of HSV-1 and incubated with supernatants containing 5 μg/ml of Mordenite, particle size 0.1-0.5 μm.

### Example 11

### Testing of Antiviral Activities of Synthetic Zeolites by the Viral Plaque Inhibition Assay—the Kinetics of Antiviral Activity of Zeolites in Cells Infected with HSV In Vitro

We further investigated the ability of synthetic zeolites to inhibit replication of HSV-1 and HSV-2 in permissive cells and cell lines.

Permissible cells were a) preincubated with different zeolites in different concentrations (as described earlier) at the 20, 16, 12, 8, 6, 4, and 2 hours before infection with HSV-1 or HSV-2; b) the zeolite was coincubated with virus, or c) was added. to the cells at 2, 4, 6, 8, 12, and 24 hours after infection. Virus was added to the cells each day for three days so the final concentration of virus in medium was always 0.1, 0.5, 1, 2 or 4 MOI (Table 4, Table 5, Table 6).

TABLE 4
A representative experiment demonstrating the decrease in percentage of number of HSV-2 plaques in
keratinocyte monolayer preincubated or postincubated at different time points with 5 μg/ml of
Mordenite or zeolite P (particle size 0.5-5 μm), and infected with 0.1, 0.5, 1, 2 or 4 MOI of HSV-2.

| | Preincubation | 0.1 | 0.5 | 1 | 2 | 4 | Co/Postincuba- | 0.1 | 0.5 | 1 | 2 | 4 |
|---|---|---|---|---|---|---|---|---|---|---|---|---|
| | time/HSV-2 | MOI | MOI | MOI | MOI | MOI | tion time/HSV-2 | MOI | MOI | MOI | MOI | MOI |
| Mordenite | −20 | 21 | 24 | 20 | 25 | 27 | 0 | 82 | 80 | 80 | 59 | 41 |
| | −16 | 32 | 40 | 42 | 40 | 35 | 0 V | 3 | 2 | 2 | 1 | 0 |
| | −12 | 63 | 67 | 62 | 55 | 41 | 0 Z + V | 88 | 90 | 83 | 70 | 54 |
| | −8 | 85 | 87 | 89 | 72 | 33 | +2 | 80 | 75 | 78 | 52 | 32 |
| | −6 | 82 | 85 | 87 | 72 | 32 | +4 | 79 | 75 | 79 | 68 | 34 |
| | −4 | 78 | 80 | 89 | 55 | 25 | +6 | 79 | 87 | 89 | 72 | 30 |
| | −2 | 81 | 83 | 83 | 61 | 30 | +8 | 65 | 60 | 52 | 43 | 19 |
| | | | | | | | +12 | 40 | 39 | 36 | 23 | 15 |
| | | | | | | | +24 | 20 | 21 | 16 | 18 | 12 |
| Zeolit eP | −20 | 19 | 22 | 21 | 15 | 10 | 0 | 59 | 50 | 52 | 29 | 18 |
| | −16 | 22 | 20 | 18 | 19 | 15 | 0 V | 2 | 3 | 2 | 1 | 0 |
| | −12 | 30 | 30 | 28 | 20 | 12 | 0 Z + V | 64 | 55 | 58 | 34 | 22 |
| | −8 | 42 | 39 | 35 | 21 | 11 | +2 | 55 | 53 | 40 | 32 | 18 |
| | −6 | 50 | 48 | 38 | 30 | 18 | +4 | 43 | 40 | 35 | 25 | 18 |
| | −4 | 55 | 55 | 47 | 31 | 20 | +6 | 35 | 29 | 29 | 19 | 12 |
| | −2 | 62 | 55 | 50 | 30 | 20 | +8 | 18 | 22 | 20 | 19 | 11 |
| | | | | | | | +12 | 18 | 18 | 16 | 12 | 12 |
| | | | | | | | +24 | 9 | 11 | 11 | 10 | 6 |

Some cultures were coinfected with the virus. Some cultures contained 10 w % of ascorbic acid, 0.01 w. % of vitamin E, 1 w. % vitamin A and D3 (V in Table), or HSV-2, zeolite and vitamins (Z + V). The antiviral effect on HSV-2 infected cells was calculated as a decrease in number of viral plaques developed after 3 days in cultures. No plaques were detected in cell controls. In cells treated with inactivated zeolites, the number of developed plaques was not statistically different from viral control. The percentage of decrease of HSV-2 plaques in acyclovir controls was between 61% (0.5 MOI at −8 hours) and 17% (4 MOI at −2 hours).

TABLE 5
A representative experiment demonstrating the decrease in percentage of number of HSV-2 plaques in
keratinocyte monolayer preincubated or postincubated at different time points with 5 μg/ml of zeolite
A or or zeolite X (particle size 0.5-5 μm) and infected with 0.1, 0.5, 1, 2 or 4 MOI of HSV-2.

| | Preincubation | 0.1 | 0.5 | 1 | 2 | 4 | Co/Postincuba- | 0.1 | 0.5 | 1 | 2 | 4 |
|---|---|---|---|---|---|---|---|---|---|---|---|---|
| | time/HSV-2 | MOI | MOI | MOI | MOI | MOI | tion time/HSV-2 | MOI | MOI | MOI | MOI | MOI |
| Zeolit A | −20 | 13 | 12 | 16 | 18 | 12 | 0 | 56 | 50 | 45 | 32 | 18 |
| | −16 | 22 | 20 | 21 | 20 | 14 | 0 V | 0 | 2 | 1 | 1 | 2 |
| | −12 | 42 | 37 | 34 | 27 | 16 | 0 Z + V | 60 | 61 | 53 | 37 | 23 |
| | −8 | 50 | 49 | 39 | 21 | 19 | +2 | 50 | 43 | 39 | 25 | 11 |
| | −6 | 52 | 51 | 45 | 28 | 16 | +4 | 45 | 40 | 33 | 25 | 14 |
| | −4 | 54 | 48 | 46 | 32 | 19 | +6 | 42 | 33 | 30 | 22 | 11 |
| | −2 | 60 | 52 | 49 | 35 | 15 | +8 | 31 | 30 | 25 | 20 | 12 |
| | | | | | | | +12 | 18 | 21 | 18 | 16 | 9 |
| | | | | | | | +24 | 10 | 10 | 9 | 8 | 9 |
| Zeolit X | −20 | 11 | 14 | 16 | 15 | 7 | 0 | 29 | 27 | 29 | 19 | 12 |
| | −16 | 20 | 20 | 17 | 18 | 9 | 0 V | 3 | 2 | 1 | 1 | 1 |
| | −12 | 22 | 17 | 18 | 17 | 12 | 0 Z + V | 34 | 35 | 31 | 23 | 18 |
| | −8 | 34 | 28 | 22 | 20 | 12 | +2 | 22 | 19 | 20 | 18 | 11 |
| | −6 | 34 | 26 | 32 | 21 | 10 | +4 | 19 | 18 | 17 | 18 | 9 |
| | −4 | 32 | 30 | 34 | 21 | 13 | +6 | 15 | 14 | 14 | 13 | 10 |
| | −2 | 35 | 30 | 33 | 21 | 14 | +8 | 17 | 16 | 17 | 13 | 8 |
| | | | | | | | +12 | 14 | 11 | 13 | 12 | 7 |
| | | | | | | | +24 | 10 | 10 | 9 | 7 | 8 |

Some cultures were coinfected with the virus. Some cultures contained 10 w % of ascorbic acid, 0.01 w. % of vitamin E, 1 w. % vitamin A and D3 (V in Table), or HSV-2, zeolite and vitamins (Z + V). The antiviral effect on HSV-2 infected cells was calculated as a decrease in number of viral plaques developed after 3 days in cultures. No plaques were detected in cell controls. In cells treated with inactivated zeolites, the number of developed plaques was not statistically different from viral control. The percentage of decrease of HSV-2 plaques in acyclovir controls was between 67% (0.5 MOI at −8 hours) and 15% (4 MOI at −2 hours).

TABLE 6
A representative experiment demonstrating the decrease in percentage of number of HSV-1 plaques in
keratinocyte monolayer preincubated or postincubated at different time points with 5 μg/ml of
Mordenite or zeolite P (particle size 0.5-5 μm) and infected with 0.1, 0.5, 1, 2 or 4 MOI of HSV-1.

| | Preincubation | 0.1 | 0.5 | 1 | 2 | 4 | Co/Postincuba- | 0.1 | 0.5 | 1 | 2 | 4 |
|---|---|---|---|---|---|---|---|---|---|---|---|---|
| | time/HSV-1 | MOI | MOI | MOI | MOI | MOI | tion time/HSV-1 | MOI | MOI | MOI | MOI | MOI |
| Mordenite | −20 | 21 | 24 | 22 | 24 | 17 | 0 | 80 | 79 | 68 | 55 | 19 |
| | −16 | 41 | 45 | 42 | 40 | 38 | 0 V | 0 | 2 | 1 | 1 | 2 |
| | −12 | 62 | 61 | 62 | 58 | 39 | 0 Z + V | 88 | 90 | 84 | 57 | 32 |
| | −8 | 80 | 78 | 80 | 70 | 35 | +2 | 71 | 67 | 60 | 52 | 19 |
| | −6 | 85 | 83 | 84 | 68 | 37 | +4 | 66 | 52 | 43 | 40 | 22 |
| | −4 | 82 | 83 | 87 | 61 | 27 | +6 | 50 | 51 | 39 | 26 | 16 |
| | −2 | 84 | 87 | 85 | 63 | 28 | +8 | 48 | 61 | 30 | 28 | 12 |
| | | | | | | | +12 | 30 | 45 | 23 | 17 | 7 |
| | | | | | | | +24 | 16 | 15 | 12 | 10 | 10 |
| Zeolit eP | −20 | 16 | 19 | 19 | 11 | 10 | 0 | 50 | 41 | 36 | 22 | 10 |
| | −16 | 22 | 21 | 17 | 18 | 10 | 0 V | 3 | 2 | 1 | 2 | 1 |
| | −12 | 28 | 30 | 28 | 18 | 11 | 0 Z + V | 54 | 52 | 47 | 32 | 23 |
| | −8 | 31 | 39 | 32 | 17 | 10 | +2 | 41 | 36 | 28 | 18 | 12 |
| | −6 | 42 | 39 | 30 | 27 | 11 | +4 | 36 | 25 | 23 | 15 | 10 |
| | −4 | 46 | 43 | 32 | 22 | 18 | +6 | 20 | 26 | 21 | 14 | 8 |
| | −2 | 55 | 50 | 45 | 28 | 18 | +8 | 17 | 12 | 15 | 11 | 6 |
| | | | | | | | +12 | 11 | 12 | 7 | 11 | 7 |
| | | | | | | | +24 | 5 | 6 | 8 | 9 | 5 |

Some cultures were coinfected with the virus. Some cultures contained 10 w % of ascorbic acid, 0.01 w. % of vitamin E, 1 w. % vitamin A and D3 (V in Table), or HSV-1, zeolite and vitamins (Z + V). The antiviral effect on HSV-1 infected cells was calculated as a decrease in number of viral plaques developed after 3 days in cultures. No plaques were detected in cell controls. In cells treated with inactivated zeolites, the number of developed plaques was not statistically different from viral control. The percentage of decrease of HSV-1 plaques in acyclovir controls was between 67% (0.5 MOI at −8 sati) and 15% (4 MOI at −2 sata). The number of plaques in wells containing cells exposed to heat-treated zeolites (inactivated zeolites) did not differ significantly from the viral control.

TABLE 7
A representative experiment demonstrating the decrease in percentage of number of HSV-1 plaques in
keratinocyte monolayer preincubated or postincubated at different time points with 5 μg/ml of
zeolite A or zeolite X (particle size 0.5-5 μm) and infected with 0.1, 0.5, 1, 2 or 4 MOI of HSV-1.

| | Preincubation | 0.1 | 0.5 | 1 | 2 | 4 | Co/Postincuba- | 0.1 | 0.5 | 1 | 2 | 4 |
|---|---|---|---|---|---|---|---|---|---|---|---|---|
| | time/HSV-1 | MOI | MOI | MOI | MOI | MOI | tion time/HSV-1 | MOI | MOI | MOI | MOI | MOI |
| Zeolit eA | −20 | 7 | 11 | 13 | 14 | 7 | 0 | 20 | 34 | 39 | 23 | 10 |
| | −16 | 18 | 17 | 18 | 15 | 11 | 0 V | 0 | 2 | 1 | 1 | 2 |
| | −12 | 19 | 29 | 34 | 17 | 10 | 0 Z + V | 27 | 29 | 32 | 27 | 19 |
| | −8 | 23 | 34 | 36 | 18 | 7 | +2 | 25 | 30 | 36 | 21 | 12 |
| | −6 | 24 | 30 | 35 | 19 | 10 | +4 | 24 | 30 | 35 | 19 | 10 |
| | −4 | 25 | 30 | 36 | 21 | 12 | +6 | 23 | 34 | 36 | 18 | 7 |
| | −2 | 23 | 32 | 39 | 23 | 10 | +8 | 19 | 29 | 34 | 17 | 10 |
| | | | | | | | +12 | 18 | 17 | 18 | 15 | 11 |
| | | | | | | | +24 | 7 | 11 | 13 | 14 | 7 |
| Zeolit X | −20 | 9 | 11 | 10 | 12 | 11 | 0 | 18 | 19 | 17 | 15 | 11 |
| | −16 | 12 | 16 | 18 | 16 | 5 | 0 V | 2 | 1 | 0 | 0 | 1 |
| | −12 | 17 | 19 | 20 | 11 | 7 | 0 Z + V | 23 | 22 | 20 | 19 | 17 |
| | −8 | 20 | 20 | 22 | 18 | 9 | +2 | 20 | 20 | 16 | 11 | 8 |
| | −6 | 20 | 23 | 20 | 16 | 11 | +4 | 17 | 18 | 17 | 11 | 9 |
| | −4 | 23 | 21 | 23 | 17 | 7 | +6 | 15 | 14 | 13 | 11 | 12 |
| | −2 | 20 | 25 | 24 | 20 | 9 | +8 | 12 | 13 | 14 | 10 | 8 |
| | | | | | | | +12 | 8 | 9 | 7 | 8 | 4 |
| | | | | | | | +24 | 6 | 10 | 11 | 8 | 7 |

Some cultures were coinfected with the virus. Some cultures contained 10 w % of ascorbic acid, 0.01 w. % of vitamin E, 1 w. % vitamin A and D3 (V in Table), or HSV-1, zeolite and vitamins (Z + V). The antiviral effect on HSV-1 infected cells was calculated as a decrease in number of viral plaques developed after 3 days in cultures. No plaques were detected in cell controls. In cells treated with inactivated zeolites, the number of developed plaques was not statistically different from viral control. The percentage of decrease of HSV-1 plaques in acyclovir controls was between 69% (0.5 MOI at −6 hours) and 13% (4 MOI at −2 sata).

### Example 12

### Testing of Antiviral Activities of Synthetic Zeolites by the Viral Plaque Inhibition Assay—the Role of Particle Size on the Inhibition of HSV Infection In Vitro

We also examined the role of particle size of zeolite on CPE decrease in HSV-2 (Table 8) and HSV-1 (Table 9) infected cells.

TABLE 8
A representative experiment demonstrating the decrease in percentage of number of HSV-2 plaques in
keratinocyte monolayer preincubated or postincubated at different time points with 5 μg/ml of Mordenite
of particle size 0.1-0.5 μm or 0.5-5 μm, and infected with 0.1, 0.5, 1, 2 or 4 MOI of HSV-2.

| | Preincubation | 0.1 | 0.5 | 1 | 2 | 4 | Co/Postincuba- | 0.1 | 0.5 | 1 | 2 | 4 |
|---|---|---|---|---|---|---|---|---|---|---|---|---|
| | time/HSV-2 | MOI | MOI | MOI | MOI | MOI | tion time/HSV-2 | MOI | MOI | MOI | MOI | MOI |
| Mordenite | −20 | 29 | 25 | 21 | 20 | 25 | 0 | 87 | 86 | 85 | 75 | 58 |
| (particle | −16 | 52 | 50 | 45 | 40 | 27 | 0 V | 2 | 1 | 3 | 2 | 1 |
| size 0.1- | −12 | 65 | 64 | 64 | 59 | 39 | 0 Z + V | 92 | 89 | 87 | 79 | 62 |
| 0.5 μm) | −8 | 79 | 76 | 73 | 70 | 40 | +2 | 83 | 81 | 75 | 54 | 45 |
| | −6 | 86 | 80 | 80 | 72 | 41 | +4 | 81 | 79 | 70 | 63 | 46 |
| | −4 | 88 | 75 | 75 | 58 | 49 | +6 | 73 | 73 | 69 | 69 | 44 |
| | −2 | 89 | 87 | 87 | 76 | 52 | +8 | 59 | 51 | 46 | 51 | 32 |
| | | | | | | | +12 | 47 | 36 | 32 | 29 | 24 |
| | | | | | | | +24 | 32 | 32 | 30 | 20 | 24 |
| Mordenite | −20 | 21 | 18 | 21 | 20 | 18 | 0 | 79 | 77 | 77 | 70 | 46 |
| (particle | −16 | 44 | 39 | 38 | 35 | 32 | 0 V | 2 | 3 | 1 | 0 | 1 |
| size 0.5- | −12 | 55 | 55 | 60 | 50 | 40 | 0 Z + V | 83 | 79 | 75 | 72 | 55 |
| 5 μm) | −8 | 76 | 75 | 70 | 68 | 30 | +2 | 77 | 70 | 73 | 41 | 37 |
| | −6 | 79 | 79 | 75 | 70 | 31 | +4 | 72 | 65 | 58 | 46 | 39 |
| | −4 | 78 | 75 | 75 | 50 | 41 | +6 | 65 | 72 | 68 | 65 | 40 |
| | −2 | 78 | 79 | 77 | 68 | 41 | +8 | 46 | 43 | 47 | 39 | 29 |
| | | | | | | | +12 | 30 | 27 | 22 | 25 | 15 |
| | | | | | | | +24 | 15 | 14 | 13 | 10 | 8 |

Some cultures were coinfected with the virus. Some cultures contained 10 w % of ascorbic acid, 0.01 w. % of vitamin E, 1 w. % vitamin A and D3 (V in Table), or HSV-2, zeolite and vitamins (Z + V). The antiviral effect on HSV-2 infected cells was calculated as a decrease in number of viral plaques developed after 3 days in cultures. No plaques were detected in cell controls. In cells treated with inactivated zeolites, the number of developed plaques wasnt statistically different from viral control. The percentage of decrease of HSV-2 plaques in acyclovir controls was between 78% (0.5 MOI at +2 hours) and 8% (4 MOI at +12 sata).

TABLE 9
A representative experiment demonstrating the decrease in percentage of number of HSV-1 plaques in
keratinocyte monolayer preincubated or postincubated at different time points with 5 μg/ml of Mordenite
of particle size 0.1-0.5 μm or 0.5-5 μm and infected with 0.1, 0.5, 1, 2 or 4 MOI of HSV-1.

| | Preincubation | 0.1 | 0.5 | 1 | 2 | 4 | Co/Postincuba- | 0.1 | 0.5 | 1 | 2 | 4 |
|---|---|---|---|---|---|---|---|---|---|---|---|---|
| | time/HSV-1 | MOI | MOI | MOI | MOI | MOI | tion time/HSV-1 | MOI | MOI | MOI | MOI | MOI |
| Mordenite | −20 | 19 | 21 | 20 | 15 | 11 | 0 | 80 | 81 | 76 | 42 | 21 |
| (particle | −16 | 32 | 23 | 17 | 18 | 14 | 0 V | 2 | 1 | 3 | 2 | 2 |
| size 0.1- | −12 | 55 | 50 | 30 | 22 | 12 | 0 Z + V | 82 | 84 | 84 | 79 | 62 |
| 0.5 μm) | −8 | 65 | 54 | 34 | 20 | 9 | +2 | 79 | 80 | 68 | 53 | 19 |
| | −6 | 70 | 63 | 55 | 29 | 10 | +4 | 72 | 70 | 69 | 40 | 15 |
| | −4 | 72 | 65 | 60 | 33 | 12 | +6 | 73 | 70 | 68 | 30 | 17 |
| | −2 | 77 | 74 | 69 | 45 | 18 | +8 | 31 | 30 | 34 | 18 | 16 |
| | | | | | | | +12 | 20 | 10 | 11 | 11 | 12 |
| | | | | | | | +24 | 11 | 9 | 12 | 9 | 9 |
| Mordenite | −20 | 12 | 10 | 11 | 10 | 11 | 0 | 72 | 73 | 70 | 36 | 15 |
| (particle | −16 | 26 | 21 | 19 | 18 | 9 | 0 V | 1 | 2 | 1 | 0 | 0 |
| size 0.5- | −12 | 36 | 39 | 27 | 22 | 10 | 0 Z + V | 74 | 75 | 72 | 41 | 19 |
| 5 μm) | −8 | 49 | 43 | 33 | 28 | 12 | +2 | 70 | 70 | 62 | 46 | 12 |
| | −6 | 47 | 50 | 51 | 36 | 9 | +4 | 65 | 62 | 60 | 34 | 11 |
| | −4 | 54 | 50 | 48 | 32 | 12 | +6 | 68 | 66 | 59 | 25 | 8 |
| | −2 | 64 | 67 | 62 | 28 | 11 | +8 | 25 | 25 | 27 | 11 | 10 |
| | | | | | | | +12 | 15 | 7 | 8 | 7 | 11 |
| | | | | | | | +24 | 7 | 7 | 7 | 4 | 3 |

Some cultures were coinfected with the virus. Some cultures contained 10 w % of ascorbic acid, 0.01 w. % of vitamin E, 1 w. % vitamin A and D3 (V in Table), or HSV-1, zeolite and vitamins (Z + V). The antiviral effect on HSV-1 infected cells was calculated as a decrease in number of viral plaques developed after 3 days in cultures. No plaques were detected in cell controls. In cells treated with inactivated zeolites, the number of developed plaques wasnt statistically different from viral control. The percentage of decrease of HSV-1 plaques in acyclovir controls was between 72% (0.5 MOI at +2 hours) and 10% (4 MOI at +24 sata).

### Example 13

### Experiments of Anti HSV Activity of Synthetic Zeolites in the Skin Cells In Vitro

Freshly obtained skin samples from surgical operations were collected in sterile containers with growth medium, supplemented with 10 times higher concentrations of antibiotics and antifungal agents than was in growth medium, for up to 24 hours. After being aseptically removed from the containers, the skin was washed 3 times with physiological saline and cut into small (1-2 mm) pieces, digested with enzymes: 0.1% trypsin/0.25% EDTA solution for 1 hour at room temperature, followed by the treatment with Dispase II overnight at 4° C. Single cell suspension was then obtained through vigorous pippetting and three washes by centrifugation (12500 rpm/10 minute). In all experiments the percentage of viable cells was >95, as checked with the trypan blue exclusion test. The cells were then preincubated with Mordenite at −20, −16, −12, −8, −6, −4. −2 hours before the infection with HSV-1 or HSV-2, coincubated with 0.1, 0.5, 1, 2 or 4 MOI of HSV-1 or HSV-2, or postincubated with the zeolite. All treatments/incubations were with MEM supplemented with physiological saline. After one hour incubation with virus, and/or zeolite in humidified CO2 incubator, the cells were washed thoroughly with physiological saline, and incubated for another 3 days with growth medium containing 9% FCS. They were fixed with 2% paraformaldehide and stained with monoclonal antibodies for the expression of a late HSV-1 or -2 glycoprotein D (gD1 and gD2) and a immediate early protein ICP27. The cells were then examined by FACS (fluorescence activated cell sorter) for the expression of HSV proteins.

TABLE 10
A representative experiment demonstrating the decrease in the percentage of glycoprotein D expression
in keratinocyte monolayer infected with 0.1, 0.5, 1, 2 or 4 MOI of HSV-2 and preincubated or postincubated
at different time points with 5 μg/ml of Mordeniteof particle size 0.1-0.5 μm or 0.5-5 μm.

| | Preincubation | 0.1 | 0.5 | 1 | 2 | 4 | Co/Postincuba- | 0.1 | 0.5 | 1 | 2 | 4 |
|---|---|---|---|---|---|---|---|---|---|---|---|---|
| | time/HSV-2 | MOI | MOI | MOI | MOI | MOI | tion time/HSV-2 | MOI | MOI | MOI | MOI | MOI |
| Mordenite | −20 | 17 | 15 | 19 | 17 | 11 | 0 | 85 | 83 | 61 | 45 | 18 |
| (\check{c}esti\check{c}ne | −16 | 27 | 27 | 24 | 28 | 12 | 0 V | 1 | 1 | 0 | 2 | 1 |
| veli\check{c}ine | −12 | 52 | 39 | 37 | 22 | 18 | 0 Z + V | 89 | 85 | 75 | 52 | 23 |
| 0.1-0.5 μm) | −8 | 60 | 52 | 40 | 22 | 17 | +2 | 82 | 67 | 50 | 38 | 15 |
| | −6 | 69 | 61 | 47 | 23 | 13 | +4 | 67 | 51 | 52 | 42 | 14 |
| | −4 | 80 | 82 | 72 | 36 | 21 | +6 | 53 | 48 | 34 | 30 | 12 |
| | −2 | 82 | 85 | 78 | 35 | 23 | +8 | 30 | 31 | 22 | 19 | 8 |
| | | | | | | | +12 | 15 | 15 | 16 | 11 | 9 |
| | | | | | | | +24 | 9 | 7 | 11 | 10 | 9 |
| Mordenite | −20 | 15 | 13 | 15 | 10 | 11 | 0 | 71 | 63 | 61 | 32 | 18 |
| (\check{c}esti\check{c}ne | −16 | 16 | 18 | 21 | 15 | 17 | 0 V | 1 | 2 | 0 | 2 | 1 |
| veli\check{c}ine | −12 | 21 | 22 | 23 | 22 | 19 | 0 Z + V | 74 | 68 | 65 | 37 | 24 |
| 0.5-5 μm) | −8 | 32 | 39 | 36 | 16 | 19 | +2 | 59 | 42 | 36 | 22 | 11 |
| | −6 | 48 | 32 | 29 | 19 | 20 | +4 | 40 | 47 | 42 | 27 | 15 |
| | −4 | 46 | 41 | 40 | 20 | 17 | +6 | 28 | 20 | 19 | 10 | 8 |
| | −2 | 66 | 60 | 41 | 28 | 11 | +8 | 11 | 13 | 14 | 11 | 8 |
| | | | | | | | +12 | 9 | 11 | 10 | 9 | 9 |
| | | | | | | | +24 | 3 | 4 | 5 | 4 | 7 |

Some cultures were coinfected with the virus. Some cultures contained 10 w % of ascorbic acid, 0.01 w. % of vitamin E, 1 w. % vitamin A and D3 (V in Table), or HSV-2, zeolite and vitamins (Z + V). The antiviral effect on HSV-2 infected cells was calculated as a decrease in the percentage of gD expression in cells after 3 days in cultures, measured by FACS. The percentage of decrease of HSV-2 gD in acyclovir controls was between 78% (0.5 MOI at −8 hours) and 9% (4 MOI at −2 hours).

TABLE 11
A representative experiment demonstrating the decrease in the percentage of ICP27 expression in keratinocyte
monolayer infected with 0.1, 0.5, 1, 2 or 4 MOI of HSV-2 and preincubated or postincubated at different
time points with 5 μg/ml of Mordenite of particle size 0.1-0.5 μm or 0.5-5 μm.

| | Preincubation | 0.1 | 0.5 | 1 | 2 | 4 | Co/Postincuba- | 0.1 | 0.5 | 1 | 2 | 4 |
|---|---|---|---|---|---|---|---|---|---|---|---|---|
| | time/HSV-2 | MOI | MOI | MOI | MOI | MOI | tion time/HSV-2 | MOI | MOI | MOI | MOI | MOI |
| Mordenite | −20 | 20 | 12 | 17 | 11 | 11 | 0 | 77 | 75 | 77 | 51 | 18 |
| (particle | −16 | 17 | 20 | 21 | 20 | 17 | 0 V | 2 | 0 | 1 | 1 | 0 |
| size 0.1- | −12 | 30 | 27 | 25 | 22 | 18 | 0 Z + V | 79 | 77 | 70 | 58 | 24 |
| 0.5 μm) | −8 | 51 | 45 | 32 | 20 | 20 | +2 | 67 | 50 | 53 | 40 | 11 |
| | −6 | 62 | 56 | 41 | 36 | 25 | +4 | 55 | 51 | 45 | 29 | 12 |
| | −4 | 70 | 65 | 62 | 45 | 23 | +6 | 32 | 32 | 23 | 30 | 11 |
| | −2 | 72 | 67 | 65 | 40 | 20 | +8 | 20 | 21 | 19 | 21 | 17 |
| | | | | | | | +12 | 11 | 12 | 14 | 13 | 10 |
| | | | | | | | +24 | 9 | 10 | 9 | 10 | 8 |
| Mordenite | −20 | 11 | 18 | 21 | 20 | 18 | 0 | 70 | 71 | 55 | 37 | 17 |
| (particle | −16 | 18 | 21 | 20 | 15 | 12 | 0 V | 0 | 1 | 1 | 0 | 1 |
| size 0.5- | −12 | 35 | 55 | 40 | 33 | 21 | 0 Z + V | 75 | 73 | 61 | 38 | 21 |
| 5 μm) | −8 | 60 | 75 | 70 | 43 | 26 | +2 | 70 | 55 | 49 | 32 | 10 |
| | −6 | 67 | 65 | 58 | 40 | 32 | +4 | 55 | 53 | 40 | 27 | 10 |
| | −4 | 62 | 75 | 75 | 47 | 18 | +6 | 39 | 27 | 22 | 20 | 9 |
| | −2 | 67 | 71 | 64 | 40 | 28 | +8 | 20 | 18 | 17 | 10 | 8 |
| | | | | | | | +12 | 10 | 6 | 6 | 8 | 10 |
| | | | | | | | +24 | 8 | 5 | 8 | 7 | 8 |

Some cultures were coinfected with the virus. Some cultures contained 10 w % of ascorbic acid, 0.01 w. % of vitamin E, 1 w. % vitamin A and D3 (V in Table), or HSV-2, zeolite and vitamins (Z + V). The antiviral effect on HSV-2 infected cells was calculated as a decrease in the percentage of ICP27 expression in cells after 3 days in cultures, measured by FACS. The percentage of decrease of ICP27 expression in acyclovir controls was between 67% (0.5 MOI at 0 hours) and 12% (4 MOI at +24 sata).

Similar results were obtained with HSV-1.

1. Synthetic zeolite for use in antiviral therapy where daily doses contain 5×10⁻⁹-1×10⁻³ g of synthetic zeolite.

2. The synthetic zeolite according to claim 1, wherein the synthetic zeolite is chosen from the group consisting of zeolite A, zeolite, X, zeolite P, and zeolite Mordenite.

3. The synthetic zeolite according to claim 2, wherein synthetic zeolite is Mordenite.

4. The synthetic zeolite according to claim 1, wherein synthetic zeolite has crystal size in a range of 0.1 micrometer-10 micrometers.

5. The synthetic zeolite according to claim 1, wherein sodium ions of the synthetic zeolite can be partially or completely exchanged with other cations.

6. The synthetic zeolite according to claim 5, wherein cations are chosen from the group consisting of K⁺, Ag⁺, NH4⁺, Ca²⁺, Mg²⁺, Zn²⁺, Cu²⁺, Fe²⁺ and Fe³⁺.

7. The synthetic zeolite according to claim 1, for prophylaxis, therapy, pre- and post-treatment of diseases caused by the infections with Herpes simplex virus type 1 and/or Herpes simplex virus type 2.

8. Pharmaceutical composition comprising synthetic zeolite or mixture thereof as active agent, pharmaceutically acceptable carrier and additives for use in antiviral therapy, where daily doses contain 5×10⁻⁹-1×10⁻³ g of synthetic zeolite.

9. The pharmaceutical composition comprising synthetic zeolite or mixture thereof as active agent, pharmaceutically acceptable carrier and additives for use in antiviral therapy according to claim 8, where the weight ratio between the active substance and the carrier is 5 ng-1 mg of active substance per one gram of carrier.

10. The pharmaceutical composition according to claim 9, wherein the zeolite is chosen from the group consisting of zeolite A, zeolite, X, zeolite P, and zeolite Mordenite.

11. The pharmaceutical composition according to claim 10, wherein synthetic zeolite is Mordenite.

12. The pharmaceutical composition according to claim 8, wherein synthetic zeolite has crystal size in a range of 0.1 micrometer-10 micrometers.

13. The pharmaceutical composition according to claim 8, wherein sodium ions of the synthetic zeolite can be partially or completely exchanged with other cations.

14. The pharmaceutical composition according to claim 13, wherein cations are chosen from the group consisting of K⁺, Ag⁺, NH4⁺, Ca²⁺, Mg²⁺, Zn²⁺, Cu²⁺, Fe²⁺ and Fe³⁺.

15. The pharmaceutical composition according to claim 8, wherein carrier is chosen from the group consisting of organic gel, water, oil, cream, liposome and liposome-based system with regular and/or extended activity.

16. The pharmaceutical composition according to claim 8, wherein additives are chosen from the group consisting of vitamins C, E, A and D3 and mixtures thereof.

17. The pharmaceutical composition according to claims 8 for prophylaxis, therapy, pre- and post-treatment of diseases caused by the infections with Herpes simplex virus type 1 and/or 2.

18. A method for topically treating a skin against virus infections, said method consisting of topical administration of the pharmaceutical composition containing 5×10⁻⁹-1×10⁻³ g of synthetic zeolite per one day.

19. The method according to claim 18, where the weight ratio between the active substance and the carrier is 5 ng-1 mg of active substance per one gram of carrier.

20. The method according to claim 18, where virus infection are infections by Herpes simplex virus type 1, and Herpes simplex virus type 2.
